(19) United States
(12) Patent Application Publication
(10) Pub. No.: US 2006/0128018 A1
(43) Pub. Date: Jun. 15, 2006

(54) Directed genetic modifications of human stem cells

(76) Inventors: Thomas P. Zwaka, Madison, WI (US); James A. Thomson, Madison, WI (US)

Correspondence Address: Nicholas J. Seay;Quarles & Brady LLP, P O Box 2113, Madison, WI, 53701-2113, US

(21) Appl. No.: 10/774,122

(22) Filed: Feb. 6, 2004

Related U.S. Application Data
(60) Provisional application No. 60/445,606, filed on Feb. 7, 2003.

(51) Int. Cl. C12N 15/87; C12N 5/08; C12N 15/85

(52) U.S. Cl. 435/455; 435/366

(57) ABSTRACT

Human embryonic stem cells can be genetically transformed by a combination of electroporation and homologous recombination. This technique makes it possible to create targeted inserts or deletions to the genome of the stem cells. This ability makes it possible to create populations of progeny cells which have differentiated into a target cell type of a specific desired lineage.

### CROSS-REFERENCE TO RELATED APPLICATIONS

This application claims priority from U.S. Provisional Patent Application No. 60/445,606 filed Feb. 7, 2003.

### STATEMENT REGARDING FEDERALLY SPONSORED RESEARCH OR DEVELOPMENT

This invention was made with United States government support awarded by the following agency: NIH RR15376. The United States has certain rights in this invention.

### BACKGROUND OF THE INVENTION

Stem cells are cells maintained in culture in vitro and which are capable of differentiation into many different differentiated cell types of a mature body. Human embryonic stem cells are a category of stem cells created originally from human embryos and are capable of indefinite proliferation in culture. Human embryonic stem cells are demonstrably pluripotent, meaning that they can differentiate into many cell types of the human body, and may be totipotent, meaning that they may be capable of differentiating into all cell types present in the developed human body.

Pluripotent embryonic stem cells have also been developed for a number of animals species other than humans. For example, much scientific work has been conducted with murine stem cells. Once techniques for the initiation and maintenance of stem cell culture for a particular species becomes known, it then becomes possible to use those stem cells to study the genetics of that species. It is now possible manipulate stem cells in a variety of ways to learn useful information about the genetics of the animal species being studies. For example, techniques have been developed over the past decade which begin with cultures of murine stem cells in which one or another specific native murine gene is rendered inactive or “knocked out.” Since murine stem cells can be successfully and ethically developed to be whole adult mice, this technique has made it possible to create strains of “knock-out” mice in which each individual strain of knockout mouse has a single gene which has been rendered defective, or “knocked out” by direct genetic manipulation. Such knock-out mice often reveal the function of a knocked-out gene because the mice are abnormal in one or more attributes which may be readily evident or which may occur only under a particular condition. The knock-out mouse technique is an important contributor to the effort to identify the function of mammalian genes in general.

It has been previously proposed that human embryonic stem cells can be transfected by a variety of techniques. Published PCT patent application WO 02/061033 describes some of that work. In that published patent application, it is reported that the most abundant gene expression activity was achieved using a transfection method based on cationic polymers, including polymers of ethyleneimine. Other techniques were found to be less effective and not preferred by that group. That work used expression vectors for exogenous genes constructed to be expressed in human cells in culture. No effort was reported in that published application to alter the genetics or the expression of native human genes in stem cells.

### BRIEF SUMMARY OF THE INVENTION

The present invention is summarized in that a method has been developed which creates directed homologous recombination events at specific targeted sites in the genome of human embryonic stem cells in culture, thus permitting the creation of human stem cells which have targeted genetic transformations in them. The genetic transformations can be knock-outs, in which the function of a particular gene is disrupted, or can be knock-ins in which the function of a particular gene is enhanced or increased or made to occur upon particular stimuli.

The present invention is also summarized in that a flexible targeted method has been developed to insert genetic constructs into targeted locations in the human genome in human stem cells in culture. This method combines the technique of homologous recombination for site direction, with electroporation, for insertion of the construct.

This invention permits directed inserts or disruptions into the genome of humans stem cells in culture and hence provides a powerful new tool to investigate the basic functioning of human genes. This technique can also be used to direct the differentiation of stem cells into specifically selected progeny cell types, thus permitting investigations into basic developmental biology of human cells.

The present invention is also directed to a method for the purification of cells of any selected lineage from human embryonic stem cells. By inserting genes into specific locations within the genome, it becomes possible to screen colonies of cells for their lineage or state of differentiation so that the purification of cells of a desired lineage or state of differentiation is possible.

The present invention is also about purifying cells of desired lineages generally. Because the method permits the purification of cells of defined lineages, it then becomes possible to characterize the molecular markers of cells of that lineage and to use those markers to purify cells of that lineage from other mixed populations of cells.

Other objects, advantages and features of the present invention will become apparent from the following specification when taken in conjunction with the accompanying drawings.

### BRIEF DESCRIPTION OF THE SEVERAL VIEWS OF THE DRAWINGS

FIG. 1 illustrates the site of gene insertion of the OCT4 genetic construct used in the examples below.

FIG. 2 is a schematic illustration of the HPRT-targeted gene vector compared to the native gene.

FIG. 3 illustrates the construction of the gene targeting vector for the human TH gene.

FIG. 4 illustrates the vector manipulations for the genetic construct for insertion of the TH gene in human ES cells.

### DETAILED DESCRIPTION OF THE INVENTION

It is revealed here for the first time that it is possible and practical to create targeted genetic transformations in primate and human embryonic stem (ES) cells through techniques based on homologous recombination events. The availability of this tool of targeted genetic transformations in human ES cells enables the purification of cells of specific desired lineage or state of differentiation, by inserting lineage or differentiation specific genetic elements into the cells. This, in turn, enables the development of a general method to purify or isolate cells of defined lineage or state of differentiation from any mixed population of cells derived from ES cells.

Targeted Gene Delivery

To achieve targeted, as opposed to random, delivery of a genetic construct into the genome of ES cells, it is necessary to rely on homologous recombination to target the delivery. To accomplish the objective of making and identifying homologous recombination events in human ES cells, a transfection technique was needed that was efficient enough to permit the identification and recovery of cells in which the homologous recombination events has occurred. Since homologous recombination events can sometimes occur at low frequencies, relatively high efficiency in the transfection method was needed so that large numbers of cells could be conveniently transfected at reasonable efficiencies. The developments of a new transfection technique was necessitated because the methods used to cause genetic transformations in murine stem cells, i.e. those techniques used to create knock-out mice, did not prove to work at sufficiently reasonable efficiencies in human embryonic stem cells. Highly stable transfection efficiencies in human embryonic stem cells have been difficult to achieve, because the electroporation protocols used for murine embryonic stem cells do not work well for human embryonic stem cells. Various research groups have reported attempts to transform human ES cells with liposome-based techniques, which are reported to work, although at apparently very low efficiencies. What is described here is a successful gene targeting methodology which makes use of homologous recombination, in conjunction with a modified electroporation technique, and that combination has proved effective at reasonable efficiency to achieve directed genetic transformations of human embryonic cell lines.

Two important attributes of the method described below are the use of electroporation to introduce the genetic construct into the ES cell and homologous recombination to facilitate introduction of the genetic construct into a desired target location in the genome of the ES cells. The use of the modified electroporation technique described below permits ES cells to be transfected by foreign DNA at reasonable efficiencies. This technique has been modified from the technique used with murine embryonic stem cells, and achieves better results in human and primate ES cells than can be achieved with the murine technique. It is demonstrated here that electroporation with homologous recombination can be used in human ES cells to achieve directed or targeted gene insertion in living human ES cells. Homologous recombination events offer a distinct advantage over random gene insertions in that the site of the insertion of foreign DNA can be controlled, thus avoiding unwanted gene insertion and permitting targeted manipulation of native genes.

To be useful in the method described here, the genetic construct should include homologous arms and a delivered genetic insert. There should be two such homologous arms, 3′ and 5′ homologous arms. The 3′ and 5′ homologous arm segments or regions are constructed to be identical in sequence to native genomic DNA sequences in regions of the genome 3′ and 5′ of the location where the genetic insert is to be inserted. In this way, by native cellular processes, the 3′ and 5′ homologous arms recombine with the corresponding native segment of DNA in the target site in the genome, thereby transferring into the genome the delivered genetic insert and removing the native DNA between the 3′ and 5′ native genomic segments. This process happens naturally using native cellular factors, but at low frequency.

The delivered genetic insert in the genetic construct that is transfected into human ES cells by the technique described here can be either a genetic insert intended to express a gene product in the ES cells, or a genetic insert which is not intended to produce a gene product. If it is desired to product a cell line in which a selected native gene in the ES cell line is silenced or disrupted, this can be done by making a “knock-out” genetic construct. In this alternative, the delivered genetic insert can be, in essence, no DNA at all, but the knock-out insertion is preferably a DNA sequence which simply does not encode a gene product at all.

If the genetic insert is intended to produce a gene product, the genetic insert should be a construction capable of expressing a gene product in an ES cell. This alternative is sometimes referred to here as the “knock-in” approach, by which a previously constructed genetic insert, producing a gene product, is substituted for a genetic sequence previously in the cells. The gene product would typically be a protein, but the production of other gene products such as RNAs (including interfering RNAs and antisense RNAs) is also contemplated. To produce a gene product, the genetic insert would typically be an expression cassette including, in sequence, a promoter, a coding sequence for the gene product and a transcriptional terminator sequence, all selected to be effective in the ES cells and appropriate for the overall process being performed.

The techniques described here are generally useful for making many kinds of targeted genetic transformations in successor cell cultures or populations made from primate and human ES cells in culture. As mentioned, this technique can be used to make either “knock-out” or “knock-in” stem cell cultures. In knock-out cells, the functioning of a particular targeted native gene is disrupted or suppressed in the genome of those cells, in order to study the effect that the lack of expression of that gene has on the viability, health, development or differentiation of the ES cells and their progeny. This is done by replacing the native genetic sequence by homologous recombination with a genetic sequence that does not express the same protein or nucleotide as the sequence replaced. Knock-out stem cells cultures of murine stem cells can be grown into so-called “knock-out mice” which have been very influential in the identification of gene function information for many genes in mice. Knock-out ES cell lines can be used to identify genes responsible for the undifferentiated status of ES cells, as well as to identify and study the function of those genes which activate the differentiation process. Knock-out cells can be useful for drug testing studies as well.

The knock-in alternative also offers a powerful way to study both gene expression and the differentiation process, as well as offering the ability to create cultures of differentiated cells directly from primary ES cells. To do this, preferably the expression cassette in the genetic insert includes a promoter which drives the expression of a screenable marker gene or selectable marker gene coding sequence which is positioned behind the promoter in the genetic construct. The promoter is a tissue specific promoter that only drives expression of the screenable or selectable marker if the ES cell into which the expression cassette has been transformed has then later differentiated into a selected cell lineage. For example, if the promoter is specific to cardiomyocytes, or heart cells, the promoter would become active to drive its associated gene expression only in those ES derived cells which have differentiated into cardiomyocytes. If the gene driven by the tissue specific promoter is a selectable marker, it can be used to select for cells which have undergone the desired differentiation. An alternative strategy is to make gene expression construct without promoters of any kind, and then to insert the construct into the genome of ES cells in a site where the genetic construct will only be expressed by native promoter activity in the cells which is specific to a desired state lineage or state of differentiation. This promoter activity would be chosen to be a promoter which is active only when the cells are in a desired differentiation lineage. Again, a screenable marker or selectable marker gene coding sequence is useful to distinguish the cells which have achieved the selected state of differentiation from other cells in culture. A screenable marker gene would be a gene the expression of which can be observed in a living cell, such as the green fluorescent protein (GFP) or luciferase, but which cannot be used to kill non-transformed cells. A screenable marker gene is used to identify transformed cells expressing the marker through visible cell selection techniques, such as fluorescent cell sorting techniques. A selectable marker would be a gene that confers resistance to a selection agent, such as antibiotic resistance, which is lethal to cells not having the selectable marker. A selectable marker is used in conjunction with a selection agent to select in culture for cells expressing the inserted gene construct.

The ability to use homologous recombination to target the delivery of genetic constructs into specific locations in the genome of human and primate ES cells is of general usefulness in permitting the expression of foreign genes or the suppression of native genes in such cells. For example, the development of techniques for creating knock-out ES cell populations, using the techniques described here, permits the creation of ES cell lines that have their native major histocompatibility (MHC) genes rendered inactive. In essence, the human MHC gene function can be knocked-out. Cells transformed in this fashion would not then present antigen on their cell surface using the MHC system. EC cells lacking MHC function would be candidate cell lines from which to develop transplantable cells or tissues, since they would presumably not engender an immune response or rejection in a host into which they were transplanted.

Prior to using electroporation, we did explore the use of chemical agents to mediate transfection of human ES cells. Those efforts did not yield satisfactory results. We also used electroporation protocols for typical mouse ES cells, such as electroporation at 220 V, 960 μF, with an electroporation medium of phosphate buffered saline, PBS, but the results were a stable transfection rate of less than 10⁻⁷. This transformation frequency experienced in human ES cells was too low to make practical the search for homologous recombination events in human ES cells. Since human ES cells are significantly larger than mouse ES cells, varying the parameters of the electroporation process was tried. Also, since the normal current culture techniques allow only about 1% of individual human ES cells to survive and form colonies when cultured at low densities, we electroporated human ES cells in clumps, not as individualized cells, and then plated out the resultant cells at high densities. Additionally, we electroporated the cells in an isotonic, protein-rich medium (standard ES cell culture medium) instead of phosphate buffered saline (PBS), used in protocols with murine cells, at room temperature. This protocol yielded G418-resistant transfection rates with human ES cells that were 100-fold (or more) higher than those that we observed using the standard protocols for mouse ES cells on human ES cells.

After one inserts a genetic construct into cells in culture, it is also possible that one may want later to remove that same insert. For example, if one inserts a genetic construct, as described here, to help identify a differentiated cell population from ES cells using a GFP marker gene, then once the differentiated cell population has been created, it may also become desirable to delete the marker gene from those cells to avoid interaction between the GFP and whatever experiment or process is to be performed with the differentiated cells. Such targeted deletions can most easily be accomplished by providing a mechanism in the genetic construct originally inserted into the ES cells which permits its ready excision. For example, the Cre/Lox genetic element could be used. The Lox sites could be built into the genetic construct transfected into the ES cells. Then if it is desired to remove the construct from the differentiated cells, the Cre agent can be added to the cells to cause the insertion to be deleted from the cells. Other similar systems may also be used.

The techniques described here for the targeted delivery of genetic constructs into human ES cells enable research to be conducted on the fundamental molecular biology of embryonic and undifferentiated cells more directly than ever possible before. One can now introduce targeted gene alterations in human embryonic stem cells in culture making, for example, targeted gene insertions or point mutations in native genes. These techniques also enable the creation of purified populations of cells of selected lineages or states of differentiation, as will be discussed next.

Lineage Purification

The genetic manipulation techniques described here can be used to direct the differentiation of primate and human ES cells into specifically desired developmental lineages. To obtain differentiated cells in general from human ES cells, it is generally not necessary to force the differentiation of ES cells in culture. In fact, primate and human ES cells, if they are permitted to have significant contact with each other, will spontaneously begin to aggregate into clumps and begin the differentiation process. To maintain the ES cells in culture in an undifferentiated state requires active effort to inhibit differentiation in order for the ES cell culture to remain in an undifferentiated form until differentiation is desired. For the directed differentiation process contemplated here, the ES cells are maintained undifferentiated until the transfection process has been performed. After transfection, the transfected ES cells are permitted to differentiate. The differentiation process would normally involve the development of ES cells into differentiated progeny successor cells of many different differentiated cell types or lineages. Even without genetic manipulation, the differentiation process can be manipulated to favor the development of one kind of successor cell or another, but this process is not highly controlled. By not highly controlled, it is meant that while the culture conditions can be manipulated to favor a particular lineage or type of differentiated progeny cell, other cell types will also develop in the culture. Thus, even if the differentiation process is directed to favor a certain cell lineage, the differentiation process will typically involve the differentiation of ES cells into a number of successor cell types. If the genetic construct introduced into the ES cells prior to differentiation includes a screenable or selectable marker, and if the genetic construct is expressed only in cells of the desired lineage or state of differentiation, the expression of the marker gene or selectable gene can then be used to identify the differentiated progeny cells of interest. As one example, if a marker gene of green fluorescent protein (GFP) is used, and if the marker gene is driven by a promoter which activates expression of the GFP gene only in a desired differentiated cell type, after differentiation the desired differentiated cells can be identified by optical cell sorting techniques (e.g. fluorescence activated cell sorting or FACS) to create populations of cells of the desired differentiated successor cell type. Thus the ability to perform sited directed insertions of genetic constructs into the genome of ES cells also permits the generation of differentiated cell populations in a directed fashion.

The ability to screen for and detect cells of a desired lineage then makes possible the purification of cultures of cells of the desired lineage. Using the GFP gene as a screenable marker for example, the GFP gene is introduced into ES cells under the control of a promoter which is specific to a desired cell lineage. Then the ES cells are permitted to differentiate, preferably under conditions which favor differentiation into the lineage sought. Then a fluorescence cell sorting device is used to sort cells for fluorescence resulting from the expression of the GFP gene. The population of cells which is selected for expression of the GFP protein will be purified for the lineages sought. By purified, it is not meant that all of the cells in the culture will be of the desired lineage. Given the efficiencies of cells sorting technology, and variations in levels of gene expression and other biological effects, some of the cells in the purified population may not be of the desired lineage. However, at a practical level, the cell culture will be purified for the lineage sought, and purified cultures of cells of specific lineages, derived from ES cells, now becomes a practical reality. Note that the lineage sought could also be undifferentiated cells, and this technique can be used to recursively selected undifferentiated cells to maintain a purified population of undifferentiated cells as well.

In fact, in one of the examples described below, this overall genetic insertion technique was used to create a marker active for undifferentiated ES cells. If one thinks of the marker system as allowing cells of a desired type of differentiation to be selected, undifferentiation can be considered as a type of differentiation. The example below uses a promoterless genetic construct which is inserted into the Oct4 gene site in the genome of the ES cells. The Oct4 gene is a member of a family of transcription factors expressed only in undifferentiated cells. The genetic construct also included a selectable marker gene (neomycin resistance) so that both antibiotic resistance and fluorescence screening could be used to identify the cells which acquired the genetic construct. The transfection efficiencies achieved, using the method described below, were better than those achieved by other methods. The transfection process performed on 1.5×10⁷ cells with a linearized vector resulted in 103 drug resistant colonies of cells. PCR analysis of the colonies revealed that 28 of the colonies, or 28%, were positive for the desired homologous recombination event. Using other vectors with longer 3′ homologous arms, the ratio could be increased to almost 40%. A test for rate of stable transfection, using a constitutive promoter, revealed a ratio of 26:1 between stable transfected clones and homologous recombination events.

Another part of the experimental work described targeted the hypoxanthine phosphoribosyltransferase gene HPRT. The HRPT gene is located on the X chromosome, so a single homologous recombination event disrupting this gene leads to complete loss of function in XY cells. In humans, mutations of this gene are found in patients having Lesch-Nyhan syndrome, a neurological disorder. Cells which are deficient in HPRT activity can be selected based on their resistance for 6-thioguanine (also referred to as 2-amino, 6 mercaptopurine) (6-TG) (Sigma cat. No. A4660), and thus the frequency of homologous recombination events can be directly estimated. It was these properties that led the HPRT gene to be used in the initial development of homologous recombination techniques in mouse cells. Doetschman, Nature 330, 576-578 (1987). The HPRT-targeted vector used here contained a short homologous arm (1.9 kb) 5′ of exon 7 and a long homologous arm (10 kb) 3′ of exon 9 of the human HPRT gene, this recombination deleting regions of the last three exons of the gene, as illustrated in FIG. 2. A neomycin resistance cassette (NEO) was inserted between the two homologous arms, and at the end of the 3′ homologous arm, the thymidine-kinase (TK) gene was added.

Another example of a marker for specific lineage differentiation is also envisioned in the experimental work described below. The gene for tyrosine hydroxylase is used as a marker for dopaminergic neurons. Other markers for other types of lineages are also envisioned in the process of the present invention.

The availability of the first purified cultures of successor lineages of differentiated cells from ES cells makes possible the development of techniques to generally screen cell populations to make other similar cultures. The first purified cultures created as described here will be transgenic for the inserted genetic construct and it is desirable to create similar purified populations of progeny cells derived from ES cell cultures which are not transgenic. This is done as follows. After the first purified population of cells of the specific lineage is created, cells of that culture are subjected to a profiling step to characterize several cellular markers specific to cells of that lineage. This can be done any number of ways, but the most efficient ways currently for doing this are by cDNA microarray gene expression analysis and by serial analysis of gene expression (SAGE). The results of that analysis will be the identification of sets of genes which are characteristic of cells that have committed to that specific lineage. With the information about that set of genes in hand, it then becomes possible to select from those genes one or more genes (and preferably three or four genes) which express cell surface markers. The expression of those cell surface markers can then be used as a test for differentiation to the lineage. New non-transgenic cultures of ES cells can be permitted to differentiate, with or without bias toward the desired progeny lineage. Then the cell surface markers can be used to screen from the mixture of cells to purify the cells that have differentiated into the desired lineage. Thus the creation of purified populations of cells of desired progeny lineages is generally enabled by the methods described here, whether or not the cells have a genetic construct inserted in them.

### EXAMPLES

Targeting the Oct4 Gene

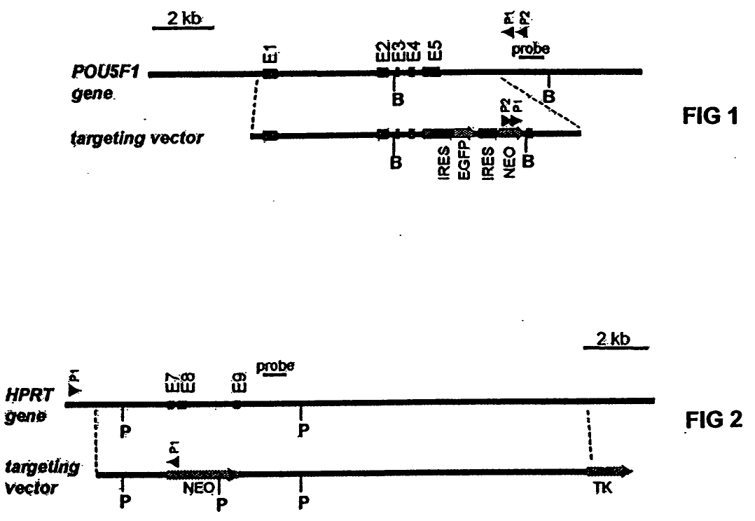

The gene targeting vector was constructed by insertion of an IRES-EGFP, an IRES-NEO, and a simian virus polyadenylation sequence (approximately 3.2 kilobases(kb)) into the 3′ untranslated region of the fifth exon of the human Oct4 gene POU5F1. This cassette is flanked in the 5′ direction by a 6.3 kb homologous arm and by a 1.6 kb (6.5 kb in the alternative targeting vector) homologous arm in the 3′ region (FIG. 1A). The cassette is inserted at position 31392 (gene accession number AC006047) of the Oct4 gene. The long arm contains sequence from 25054-31392 (gene accession number AC006047). The short arm contains the sequence from 31392-32970 (gene accession number AC006047). In the alternative targeting vector, the short arm is substituted by a longer homologous region (31392-32970 in AC006047 plus 2387-7337 in gene accession number AC004195). Isogenic homologous DNA was obtained by long distance genomic PCR and subcloned. All genomic fragments and the cassette were cloned into the multiple cloning site of pBluescript SK II. H1.1 human embryonic stem (ES) cells were cultured using human ES cell medium consisting of 80% Dulbecco's modified Eagle's medium (no pyruvate, high glucose formulation; Invitrogen) supplemented with 20% Gibco KNOCKOUT Serum Replacement, 1 mM glutamine, 0.1 mM b-mercaptoethanol (Sigma), 1% nonessential amino acid stock (Gibco) and 4 ng/ml human basic fibroblast growth factor (Invitrogen). One week before electroporation, cells were plated onto matrigel (Becton Dickinson) coated 10 cm dishes and cultured with murine embryonic fibroblast conditioned media supplemented with 4 ng/ml basic fibroblast growth factor. For electroporation, cells were harvested with collagenase IV (1 mg/ml, Invitrogen) for 7 min at 37° C., washed with medium, and resuspended in 0.5 ml culture medium (1.5-3.0×10⁷ cells). Just prior to electroporation, 0.3 ml phosphate buffered saline (PBS, Invitrogen) containing 40 mg linearized targeting vector DNA was added. Cells were then exposed to a single 320 V, 200 μF pulse at room temperature using the BioRad Gene Pulser 11 (0.4 cm gap cuvette). Cells were incubated for 10 minutes at room temperature and were plated at high density on matrigel. G418 selection (50 mg/ml, Invitrogen) was started 48 hours after electroporation. After one week, G418 concentration was doubled. After three weeks, surviving colonies were analyzed individually by PCR using primers specific for the NEO cassette and for the POU5F1 gene just downstream of 3′ homologous region, respectively. PCR positive clones were re-screened by Southern blot analysis using BamHI digested DNA and a probe outside the targeting construct.

Flow Cytometry

Prior to flow cytometry, ES cell differentiation was induced by incubating the cells for five days in unconditioned medium on matrigel. ES cells were treated with trypsin/EDTA and washed with PBS (both Invitrogen). Dead cells were excluded from analysis by forward- and side-scatter gating. Samples were analyzed using a FACScan (Becton Dickinson) flow cytometer and Cellquest software (Becton Dickinson). A minimum of 50,000 events was acquired for each sample.

Using this combination of selection by the use of the G418 antibiotic and the flow cytometry for GFP expression, undifferentiated cells were purified from a culture containing both undifferentiated cells and a mix of partially differentiated cells. The undifferentiated cells were then analysized using a cDNA microarray. The expression of several genes indicative of the status of undifferentiated cells were identified, including CD124, CD113, FGF-R, c-Kit, and BMP4-R. These markers were not previously identified as associated with human ES cells.

Next, antibodies for the identified markers will be created. The antibodies will be used to affinity purify undifferentiated cells about of mixed populations of cells to maintain purified cultures of undifferentiated cells.

Targeting the HPRT Gene

HPRT Knock-Out.

Prior experience has suggested that for human ES cells, the best chemical reagents yield stable, drug-selectable transfection rates of about 10⁻⁵. Using the electroporation techniques developed for mouse ES cells yielded even poorer efficiencies. We tested two chemical transfection reagents, ExGen 500™ and FuGene-6™ as mediators of homologous recombination events at the HPRT locus in human ES cells. Although G418 and gancyclovir-resistant clones were obtained using both transformation reagents, none of the resulting clones were 6-TG resistant, indicating that none of the clones were the results of homologous recombination. There results are consistent with the observation that transfection using lipid and cationic reagents results in inefficient homologous recombination in other mammalian cell types and that physical means of introducing DNA are generally more conducive to homologous recombination events.

The gene-targeting vector was constructed by substitution of the last three exons (exon 7, 8 and exon 9) of the HPRT gene by a NEO-resistance cassette under TK promoter control. This cassette is flanked in the 5′ direction by a 10 kb homologous arm and by a 1.9 kb homologous arm in the 3′ region (FIG. 2). Isogenic homologous DNA was obtained by long-distance genomic PCR and subcloned. Human ES cells of line H1.1 were cultured using standard hES cell culture methodologies. One week before electroporation, cells were plated onto Matrigel™ and cultured under fibroblast conditioned medium. To remove clones as intact clumps, human ES cell cultures were treated with collagenase IV (1 mg/ml, Invitrogen) for 7 min, washed with medium, and resuspended in 0.5 ml culture medium (1.5-3.0×10⁷ cells). Just prior to electroporation, 0.3 ml phosphate-buffered saline (PBS, Invitrogen) containing 40 μg linearized targeting vector DNA was added. Using the electroporation parameters mentioned above (standard ES media, ES cells in clumps) human ES cells were then exposed to a single 320 V, 200 μF pulse at room temperature using the BioRad Gene Pulser II (0.4 cm gap cuvette). Cells were incubated for 10 minutes at room temperature and were plated at high density (one 10 cm culture dish) on Matrigel. G418 selection (50 μg/ml, Invitrogen) was started 48 hours after electroporation. After one week, G418 concentration was doubled and 6-TG selection (1 mM, Sigma) was started. After three weeks, surviving colonies were analyzed individually by PCR using primers specific for the NEO cassette and for the HPRT gene just upstream of 5′ homologous region, respectively. PCR-positive clones were rescreened by Southern blot analysis using PstI-digested DNA and a probe 3′ of the NEO cassette (FIG. 2).

The result of this analysis was that after transfection of 10⁷ cells with the linearized HPRT-targeted vector, 350 G418-resistant clones were obtained. Of these, 50 were gancyclovir-resistant, and, of these, 7 were also 6-TG resistant, suggesting successful homologous recombination. Polymerase chain reaction, PCR, and Southern blotting confirmed that homologous recombination had occurred in all the 6-TG resistant clones.

The rates of successful transformation using chemical reagents and electroporation are summarized in Table 1 below.

TABLE 1
Numbers of colonies obtained by positive and negative
selection and targeted events in the HPRT gene locus
(from 1.5 × 10⁷ electroporated human ES cells)

| Selection procedure | ExGen 500 | Fugene 500 | Electroporation |
|---|---|---|---|
| G418 | 130 | 261 | 350 |
| G418 and gancyclovir | 35 | 61 | 50 |
| G418 and 6-TG | 0 | 0 | 7 |

Dopaminergic Neurons

Tyrosine hydroxylase (TH) is the rate-limiting enzyme in the synthesis of dopamine, and it is one of the most common markers used for dopaminergic neurons. Although TH is not specific for midbrain dopaminergic neurons, current ES cell differentiation protocols that use FGF8 and sonic hedgehog produce TH-positive neurons that are highly enriched for a midbrain ventral specification. However, these procedures produce TH-positive dopaminergic cells mixed with other cell types. We, therefore, decided to use TH as a marker to purify human ES cell-derived dopaminergic neurons from other cells in this mixed population of cells.

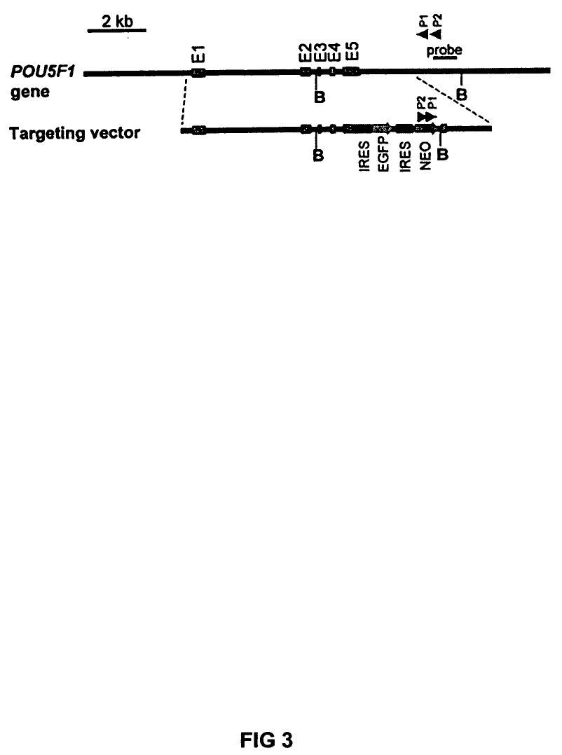

In order to achieve expression of both TH and EGFP, we constructed a gene-targeting vector that introduces an IRES-EGFP reporter gene cassette into the 3′ UTR region in the last exon of the TH gene. All further positions detailed below are given relative to the position of the stop codon of the TH gene in the DNA sequence L15440 (gene accession number 307071). The IRES-EGFP cassette (from Clontech) and loxP-PGK-NEO cassette (kindly provided by H. J. Fehling, University of Ulm, Germany) is flanked by a short homologous arm 5′ of the stop codon (exactly 1227 base pairs) and a long homologous arm in the 3′ region the stop codon (exactly 7955 base pairs). In a first cloning step these two homologous DNA arms were amplified using long distance PCR (Roche Long Distance PCR kit) and subcloned into the pGEM-Teasy vector (Promega). In the next cloning step the short arm (pT-TH-SA) was cut out using the restriction enzymes SalI and XhoI and cloned into the SalI site of pTH-AA. These manipulations are shown in FIG. 3.

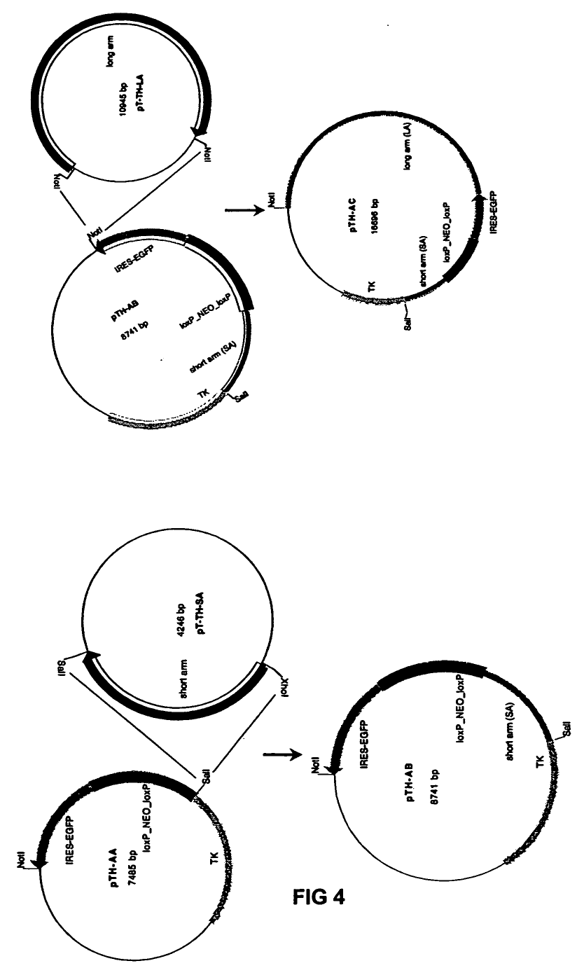

In the next cloning step the subcloned long arm is cut out of pT-TH-LA using NotI and cloned into pTH-AB using a NotI site. The long arm follows the gene coding for thymidine kinase (TK) for negative selection of random integrated, stable, transfected clones. Between the long homologous arm and the IRES-EGFP cassette, we cloned a PGK-driven NEO resistance cassette embedded between two loxP sites. FIGS. 4 and 5 depict the important elements of the gene targeting vector. After electroporation as described above, we were able to obtain five PCR and southern-blot confirmed, homologous recombinant clones after double selection for the positive selection marker NEO with G418 and the negative selection marker TK with gancyclovir.

The positive selection marker in this experiment was a NEO cassette under the PGK promoter. As this cassette is still present in the knock-in cells line, it could alter the expression level of the TH gene itself and of the IRES-EGFP reporter gene. Therefore, it is considered as a standard to delete this selection cassette. To do so we transiently transfected two of the TH-EGFP knock-in cell lines with a plasmid containing the phage recombinase Cre under the control of the EF1A promoter. The cDNA of Cre was followed by an IRES-EGFP cassette. After transient transfection with this plasmid, Cre over-expressing cells could be easily identified by EGFP expression. Those EGFP-positive cells were purified by fluorescence activated cell sorting (FACS). Individual clones were analyzed for successful recombination of the two loxP sites, and two clones were identified that had the NEO cassette excised.

We used embryoid bodies to differentiate human ES cells into neurons. Embryoid body formation and neural differentiation was performed according to the methods already described in the scientific and patent literature. Human ES cell colonies were released intact from the flask by exposure to dispase (0.1 mg/ml) for 30 min. The colonies were washed, and resuspended in ES cell medium lacking bFGF and cultured for four days in suspension. The culture was fed daily, and any attaching clumps gently dislodged. The resulting embryoid bodies were plated in a new flask, in DMEMF12 supplemented with insulin (25 mg/ml), transferrin (100 mg/ml), progesterone (20 NM), putrescine (60 mM), sodium selenite (30 mM), and heparin (2 mg/ml) in the presence of bFGF (4 ng/ml) and allowed to attach. The differentiating embryoid bodies (Ebs) were cultured for an additional 8-10 days, and neural rosette cells are separated from the surrounding flat cells by exposure to 0.1 mg/ml dispase. The resulting enriched neural rossette cells were further cultured in the presence of FGF2 (20 ng/ml), FGF8 (100 ng/ml) and sonic hedgehog (400 ng/ml) to induce midbrain, ventral dopaminergic neuron differentiation. We collected cells and determined the number of EGFP-positive cells by FACS, and the morphology of the cells was examined under the fluorescence microscope.

The knock-in cell line will be differentiated with the appropriate differentiation protocol, and at the time point of maximal GFP expression for each cell line, the cells will be subjected to FACS, and sorted based on GFP fluorescence intensity. Sorted GFP-positive and − negative cells will be analyzed by western blotting for the specific protein (TH). RNA from the population will be collected for gene expression profiling and the identification of specific cell surface proteins (cDNA microarray and SAGE).

I/We claim:

1. A method of performing targeted modifications of human embryonic stem (ES) cells, the method comprising the steps of
obtaining copies of a genetic construct which includes a foreign gene and which has regions at either of its ends homologous with a pair of selected regions in the genome of the ES cells so that homologous recombination will occur between the genetic construct and the selected regions of the genome of the stem cells;
electroporating the copies of the genetic construct into stem cell in culture; and
identifying cells which contain the genetic construct.

2. A method as claimed in claim 1 where the genetic construct include a marker gene.

3. A method as claimed in claim 2 wherein there is no promoter on the marker gene in the genetic construct, the genetic construct being inserted into the ES cells in a location in the genome of the ES cells wherein the marker gene is expressed only in cells in a desired state of differentiation.

4. A method as claimed in claim 2 wherein there is a tissue specific promoter driving the expression of the marker gene in the genetic construct, the tissue specific promoter being active only in cells in a desired state of differentiation.

5. Human cells in culture derived from human embryonic stem cells, the cells comprising in their genome an inserted genetic construct which knocks out the functioning of a gene which would otherwise be expressed in those human cells in culture.

6. Human cells in culture derived from human embryonic stem cells, the cells comprising in their genome an inserted genetic construct which introduced a mutation into a native gene in those human cells in culture.

7. A method of purifying cells of a defined lineage from cultures of human embryonic stem (ES) cells, the method comprising the steps of
obtaining copies of a genetic construct which has regions at either of its ends homologous with a pair of selected regions in the genome of the ES cells so that homologous recombination will occur between the genetic construct and the selected regions of the genome of the stem cells, the genetic construct including a marker gene which will be expressed only in cells of the defined lineage;
electroporating the copies of the genetic construct into stem cell in culture; and
identifying cells which express the marker gene in the genetic construct and purifying those cells from other cells.

8. A method as claimed in claim 7 wherein the marker gene includes a promoter which is active to express a gene only in cells of the desired lineage.

9. A method as claimed in claim 7 wherein after the electroporating step, the ES cells are permitted to differentiate.

10. A method as claimed in claim 7 wherein the marker gene expresses a fluorescent gene product and the identifying and purifying is performed by fluorescence activated cell sorting.

11. A culture of differentiated human cells derived from human ES cells and purified by the method of claim 7 for cells of a desired lineage.

12. A method for purifying cells of a defined lineage derived from human embryonic stem (ES) cells, the method comprising the steps of
purifying cells of the defined lineage by the method of claim 6;
analyzing the gene expression pattern of the purified cells to identify genes expressed in the cells of the defined lineage which are characteristic of that lineage;
culturing non-transformed ES cells so that the ES cells initiate differentiation; and
purifying the cells of the defined lineage based upon the expression of the genes identified in the analyzing step.

13. A method as claimed in claim 12 wherein the defined lineage is undifferentiated cells wherein the genes identified include genes for the cellular factors CD124, CD113, FGF-R, c-Kit, and BMP-4, and wherein the purification step is performed by testing cells for expression of at least one gene selected from the groups consisting of CD124, CD113, FGF-R, c-Kit, and BMP-4.

14. Human cells in culture derived from human embryonic stem cells, the cells comprising in their genome an inserted genetic construct which expresses an inserted gene only when the human cells are in a desired state of differentiation.

15. Human cells in culture as claimed in claim 14 wherein the desired state of differentiation is an undifferentiated state.

16. Human cells in culture as claimed in claim 14 where the gene is a marker gene the expression of which can be observed visually.
